(19) United States
(12) Patent Application Publication
(10) Pub. No.: US 2007/0016178 A1
(43) Pub. Date: Jan. 18, 2007

(54) Laser energy delivery device with swivel handpiece

(76) Inventors: Boris Vaynberg, Zichron Ya'akov (IL); Yotam Zimmerman, Hadera (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/180,546

(22) Filed: Jul. 14, 2005

(51) Int. Cl. A61B 18/18

(52) U.S. Cl. 606/19; 606/13

(57) ABSTRACT

A laser energy delivery device including a laser source operatively connected to a handpiece by means of a swivel joint, the swivel joint including a first reflector mounted in a swivel arm, one portion of the swivel arm being rotatably attached to the laser source and having a window for laser energy to pass from the laser source to the first reflector, and another portion of the swivel arm being rotatably attached to the handpiece, wherein the laser energy is directed from the first reflector to the handpiece.

### FIELD OF THE INVENTION

The present invention relates generally to laser energy delivery, and particularly to a laser energy delivery device with a swivel handpiece.

### BACKGROUND OF THE INVENTION

There are many devices in the prior art for delivering laser energy from a laser energy source to a surgical site of a patient, which employ a handpiece that can be rotated or flexed to deliver the energy to hard-to-reach places (e.g., in the mouth of a patient in dentistry applications).

Some prior art devices place the laser source in a handheld probe or handpiece. The handpiece may include a water cooling line, high power electrical line and signal lines. It may also contain additional lines, such as a water spray line. However, the laser is a relatively bulky element in the handpiece, and as such, the handpiece is usually unwieldy and uncomfortable.

As another example, U.S. Pat. No. 5,346,489 to Levy, et al., describes a medical laser delivery system that includes a handpiece adapted for rotatable coupling to a delivery arm coupled to the laser energy source such that the handpiece is rotatable about its longitudinal axis with respect to the delivery arm. A distal delivery system is coupled to the handpiece and delivers laser energy to the surgical site when the laser energy source is actuated. The handpiece includes a handpiece body adapted for rotatable coupling to the delivery arm and a handpiece head coupled to both the handpiece body and the distal delivery system. The handpiece head can be rotatably coupled to the handpiece body. In one embodiment, the handpiece head is angularly oriented at a fixed angle with respect to the handpiece body. In an alternative embodiment, the handpiece head is pivotally coupled to the body such that the handpiece head may be adjusted to various angles with respect to the handpiece body, such pivotal coupling preferably being accomplished by a ball and socket joint. A removable fiber extension is positioned within the handpiece and transmits laser energy from a fiber in the delivery arm to the distal delivery system.

### SUMMARY OF THE INVENTION

As described more in detail hereinbelow, the present invention seeks to provide an improved laser energy handpiece. In accordance with an embodiment of the invention, the handpiece may be connected to the laser source with a swivel joint including one or more reflectors.

There is provided in accordance with an embodiment of the present invention a laser energy delivery device including a laser source operatively connected to a handpiece by means of a swivel joint, the swivel joint including a first reflector mounted in a swivel arm, one portion of the swivel arm being rotatably attached to the laser source and having a window for laser energy to pass from the laser source to the first reflector, and another portion of the swivel arm being rotatably attached to the handpiece, wherein the laser energy is directed from the first reflector to the handpiece.

In accordance with an embodiment of the present invention, a second reflector may be mounted in the swivel arm, wherein laser energy is reflected from the first reflector to the second reflector and is directed through another window at another portion of the swivel joint to the handpiece.

The handpiece may be arranged for swiveling around its own longitudinal axis and/or around a longitudinal axis of the laser source.

The first reflector may be pivoted about a pivot, such that an incident angle between the first reflector and the laser source is changeable.

The handpiece may be detachable from the swivel joint. The handpiece may be disposable or sterilizable.

### BRIEF DESCRIPTION OF THE DRAWINGS

The present invention will be understood and appreciated more fully from the following detailed description, taken in conjunction with the drawings in which:

FIG. 1A is a simplified illustration of a laser energy delivery device, constructed and operated in accordance with an embodiment of the present invention; and

FIG. 1B is a simplified illustration of a laser energy delivery device, constructed and operated in accordance with another embodiment of the present invention.

### DETAILED DESCRIPTION OF EMBODIMENTS

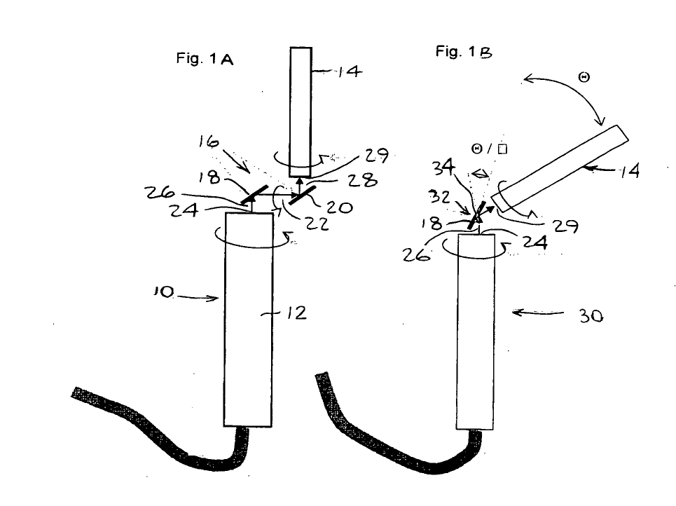

Reference is now made to FIG. 1A, which illustrates laser energy delivery device 10, constructed and operative in accordance with an embodiment of the present invention.

Laser energy delivery device 10 may include a laser head 12, also referred to as laser source 12, operatively connected to a handpiece 14 by means of a swivel joint 16. In the non-limiting embodiment of FIG. 1A, the swivel joint 16 includes a first reflector 18 and a second reflector 20 mounted in a swivel arm 22. The reflectors may be polished mirrors, for example. One portion of the swivel arm 22 is mounted for rotation on the laser source 12, such as by means of a bearing 24. This portion has a window 26 for the laser energy to pass through to the first reflector 18. The laser energy is reflected from first reflector 18 to second reflector 20 and exits another window 28 at the opposite end of swivel arm 22, connected to handpiece 14. The handpiece 14 may be rotatably connected to swivel arm 22 by means of a bearing 29. In this manner, the handpiece 14 can swivel around its own longitudinal axis and around the longitudinal axis of the laser source 12, thereby providing additional comfort and maneuverability to the user.

The size and nature of the windows 26 and 28 is determined by the laser energy being used. It is noted that the windows do not necessarily have to be physical openings; rather it is sufficient for them to be transmissive of the laser energy being used.

The first and second reflectors 18 and 20 may be angled at 45° with respect to the laser source 12 and handpiece 14, respectively, independent of the angle between handpiece 14 and laser source 12. Other angles may also be used.

Reference is now made to FIG. 1B, which illustrates laser energy delivery device 30, constructed and operative in accordance with an embodiment of the present invention. Components of laser energy delivery device 30 similar to laser energy delivery device 10 are designated by like reference numerals. Laser energy delivery device 30 differs from laser energy delivery device 10 in the construction of the swivel joint. In laser energy delivery device 30, a swivel arm 32 may include first reflector 18 (e.g., a polished mirror), and is mounted for rotation on the laser source 12, such as by means of bearing 24. Laser energy may pass through to the first reflector 18 via window 26. The laser energy is reflected from first reflector 18 directly into handpiece 14, which may be rotatably connected to swivel arm 32 by means of bearing 29. In this manner, the handpiece 14 can swivel around its own longitudinal axis and around the longitudinal axis of the laser source 12. In the embodiment of FIG. 1B, unlike the embodiment of FIG. 1A, the first reflector 18 may be pivoted about a pivot 34, such that the incident angle between the first reflector 18 and the laser source 12 is not necessarily constant, i.e., changeable. The incident angle is always equal to half the angle between the laser source 12 and the handpiece 14.

The laser source 12 may include emit a laser beam in any wavelength range, and may be pulsed or continuous wave.

In all embodiments, handpiece 14 may be detachable from the swivel joint. Handpiece 14 may be disposable or it may be sterilized (e.g., autoclaved). A variety of detachable handpieces may be provided to the end user, having different parameters, such as but not limited to, different spot sizes, different beam quality, different tips, different dimensions and shapes, etc.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. Laser energy delivery device comprising:
a laser source operatively connected to a handpiece by means of a swivel joint, said swivel joint comprising a first reflector mounted in a swivel arm, one portion of said swivel arm being rotatably attached to said laser source and having a window for laser energy to pass from said laser source to said first reflector, and another portion of said swivel arm being rotatably attached to said handpiece, wherein the laser energy is directed from said first reflector to said handpiece.

2. The laser energy delivery device according to claim 1, further comprising a second reflector mounted in said swivel arm, wherein laser energy is reflected from said first reflector to said second reflector and is directed through another window at another portion of said swivel joint to said handpiece.

3. The laser energy delivery device according to claim 1, wherein said handpiece is arranged for swiveling around its own longitudinal axis.

4. The laser energy delivery device according to claim 1, wherein said handpiece is arranged for swiveling around a longitudinal axis of said laser source.

5. The laser energy delivery device according to claim 2, wherein said first and second reflectors are angled at 45° with respect to said laser source and said handpiece, respectively.

6. The laser energy delivery device according to claim 1, wherein said first reflector is pivoted about a pivot, such that an incident angle between said first reflector and said laser source is changeable.

7. The laser energy delivery device according to claim 1, wherein the incident angle is equal to half the angle between said laser source and said handpiece.

8. The laser energy delivery device according to claim 1, wherein said handpiece is detachable from said swivel joint.

9. The laser energy delivery device according to claim 1, wherein said handpiece is disposable.

10. The laser energy delivery device according to claim 1, wherein said handpiece is sterilizable.
